(19) United States
(12) Patent Application Publication
(10) Pub. No.: US 2022/0287648 A1
(43) Pub. Date: Sep. 15, 2022

(54) Systems and Methods for Imputing Real-Time Physiological Signals

(71) Applicant: The Regents of the University of California, Oakland, CA (US)

(72) Inventors: Maxime Cannesson, Los Angeles, CA (US); Brian Hill, Los Angeles, CA (US); Eran Halperin, Santa Monica, CA (US); Ira Hofer, Los Angeles, CA (US); Nadav Rakocz, Los Angeles, CA (US)

(73) Assignee: The Regents of the University of California, Oakland, CA (US)

(21) Appl. No.: 17/753,126

(22) PCT Filed: Aug. 19, 2020
(86) PCT No.: PCT/US20/47044

§ 371 Date: Feb. 18, 2022

Related U.S. Application Data
(60) Provisional application No. 62/890,022, filed on Aug. 21, 2019.

(51) Int. Cl. A61B 5/00; A61B 5/318; A61B 5/024; A61B 5/308

(52) U.S. Cl. CPC A61B 5/7267; A61B 5/318; A61B 5/02416; A61B 5/742; A61B 5/308; A61B 5/7203

(57) ABSTRACT

Systems and methods for training a signal generation model and generating imputed physiological waveform signals in accordance with embodiments of the invention are illustrated. One embodiment includes a method for measuring physiological waveform signals. The method includes steps for receiving a set of one or more input physiological waveform signals, processing the set of input physiological waveform signals, generating an output physiological waveform signal using a signal generation model, and providing outputs based on the generated output signal.

This invention was made with government support under Grant Number HL144692, awarded by the National Institutes of Health and Grant Number 1705197, awarded by the National Science Foundation. The government has certain rights in the invention.

### FIELD OF THE INVENTION

The present invention generally relates to sequence-to-sequence transformation and, more specifically, to imputing real-time physiological signals using machine learning models.

### BACKGROUND

In various medical situations, it can be inconvenient and/or expensive to continuously monitor physiological signals. For example, in 90% of surgeries, arterial blood pressure (ABP) is monitored non-invasively but intermittently (every 3 minutes) using a blood pressure cuff. In the remaining 10%, ABP is measured continuously but invasively. Since even a few minutes of hypotension increase postoperative mortality, and because invasive monitoring is associated with major complications (infection, bleeding, thrombosis), the ideal ABP monitor should be non-invasive and continuous.

### SUMMARY OF THE INVENTION

Systems and methods for training a signal generation model and generating imputed physiological waveform signals in accordance with embodiments of the invention are illustrated. One embodiment includes a method for measuring physiological waveform signals. The method includes steps for receiving a set of one or more input physiological waveform signals, processing the set of input physiological waveform signals, generating an output physiological waveform signal using a signal generation model, and providing outputs based on the generated output signal.

In a further embodiment, receiving the set of input physiological waveform signals includes capturing the set of input signals in a non-invasive manner.

In still another embodiment, processing the set of input physiological waveform signals includes at least one of normalizing, scaling, filtering, and downsampling the set of input physiological waveform signals.

In a still further embodiment, the signal generation model is a convolutional neural network (CNN) that takes a set of windows from the set of input physiological waveform signals as input and generates a window of the output physiological waveform signal.

In yet another embodiment, providing outputs includes providing at least one of a summary statistic and a visualization of the output physiological waveform signal.

Another embodiment includes a method for training a signal generation model to generate a physiological waveform signal. The method includes steps for receiving a set of one or more input physiological waveform signals, processing the set of input physiological waveform signals, generating an output physiological waveform signal using a signal generation model, computing a loss between the generated output physiological waveform signal and a true output physiological waveform signal, and modifying the signal generation model based on the computed loss.

In a yet further embodiment, the set of input physiological waveform signals includes at least one of an electrocardiogram (ECG) and a photo-plethysmogram (PPG).

In another additional embodiment, processing the set of input physiological waveform signals includes at least one of normalizing, scaling, filtering, and downsampling the set of input physiological waveform signals.

In a further additional embodiment, the signal generation model is a convolutional neural network (CNN) initialized with a random set of weights.

In another embodiment again, computing a loss includes a penalty for the difference between maximum signal points and minimum signal points of the output and true physiological waveform signals. For example, maximum and minimum signal points can represent systolic and diastolic pressure points respectively.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 conceptually illustrates a process for generating imputed signals in accordance with an embodiment of the invention.

FIG. 2 illustrates the generation of ABP signals in accordance with an embodiment of the invention.

FIG. 3 conceptually illustrates a process for scaling input signals in accordance with an embodiment of the invention.

FIG. 4 conceptually illustrates a process for processing a set of signals in accordance with an embodiment of the invention.

FIG. 5 illustrates an example of an architecture for a signal generation model in accordance with an embodiment of the invention.

FIG. 6 conceptually illustrates an example of a process for training a signal generation model in accordance with an embodiment of the invention.

FIG. 7 illustrates a visualization of plots for the differences between the predicted and actual blood pressure measures.

FIG. 8 illustrates an example of a signal generation system that trains a model and generates signals in accordance with an embodiment of the invention.

FIG. 9 illustrates an example of a signal generation element that generates signals in accordance with an embodiment of the invention.

FIG. 10 illustrates an example of a training element that trains a signal generation system in accordance with an embodiment of the invention.

FIG. 11 illustrates an example of a signal generation application that generates signals in accordance with an embodiment of the invention.

FIG. 12 illustrates an example of a training application that trains a signal generation network in accordance with an embodiment of the invention.

### DETAILED DESCRIPTION

Turning now to the drawings, systems and methods for obtaining a sequence-to-sequence transformation to impute a set of physiological waveform signals in real-time using machine learning are described. Currently available non-invasive and continuous ABP monitors are expensive and often utilize sophisticated sensors requiring expensive production and regulatory control costs. Processes in accordance with certain embodiments of the invention can improve the current standard of care by adding non-invasive and continuous monitoring of patient ABP to the majority of surgical patients that do not undergo invasive ABP measurement. Being able to monitor ABP continuously in all patients has potential to improve postoperative outcomes because even a few minutes of hypotension during surgery leads to increased postoperative complications. While other non-invasive ABP monitoring devices are available, systems and methods in accordance with certain embodiments of the invention do not require new physical medical devices and can often be implemented in software with existing non-invasive devices. Systems and methods in accordance with a variety of embodiments of the invention can deduce an individual patient's blood pressure model from population data, without using any prior information about the patient. In many embodiments, the model does not need to be further calibrated specifically for the patient.

### Generating Waveform Signals

Systems and methods in accordance with a number of embodiments of the invention can improve the standard-of-care for patients by imputing relevant physiological waveform signals, which may be difficult to obtain, in real time by using readily-available data as input to the method. In numerous embodiments, physiological signals that can be imputed may typically be difficult to obtain due to increased health risks introduced by obtaining the signal from a patient, additional time and resources required for health providers to set up signal monitoring, and/or prohibitive costs. As opposed to summary statistics (e.g., diastolic or systolic blood pressures), real-time imputation of physiological waveforms in accordance with various embodiments of the invention can allow healthcare providers to continuously monitor a patient in situations where the imputed waveform signal would otherwise be unavailable.

For example, in 90% of surgeries, arterial blood pressure (ABP) is monitored non-invasively but intermittently (every 3 minutes) using a blood pressure cuff. In the remaining 10%, ABP is measured continuously but invasively. Since even a few minutes of hypotension can increase the risk of postoperative mortality, and because invasive monitoring is associated with major complications (infection, bleeding, thrombosis, pain) ABP monitors in accordance with a variety of embodiments of the invention can be non-invasive and continuous. In many embodiments, systems can provide non-invasive and continuous monitoring of patient ABP to the majority of surgical patients that do not undergo invasive ABP measurement.

Although many of the examples described herein describe applications in monitoring ABP, one skilled in the art will recognize that similar systems and methods can be used in a variety of applications, including (but not limited to) end-tidal CO2 monitoring, without departing from this invention.

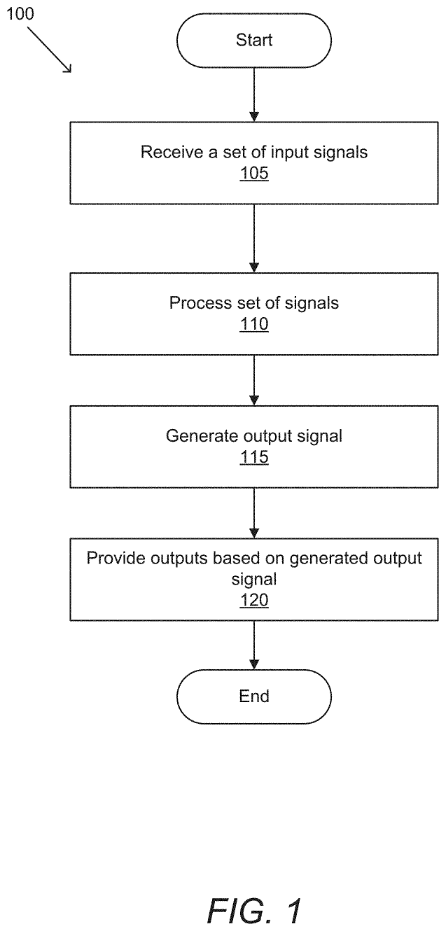

Processes in accordance with a variety of embodiments of the invention can train a model to learn a function that takes one set of physiological waveform signals (e.g., electrocardiogram (ECG), photo-plethysmogram (PPG), etc.) as input to generate a different set of physiological signals (e.g., ABP). An example of a process for generating signals in accordance with an embodiment of the invention is conceptually illustrated in FIG. 1. Process 100 receives (105) a set of one or more input signals. Input signals in accordance with many embodiments of the invention can include physiological signals that can be obtained invasively and/or non-invasively. In several embodiments, input signals are obtained non-invasively, and are used to impute signals that would traditionally be obtained through an invasive process. Input signals in accordance with several embodiments of the invention can include (but are not limited to) blood pressure, oscillometric waveforms, heart rates, central venous pressures, pulmonary artery pressure, near infrared spectroscopy signals, intra cranial pressure, electroencephalographic waveforms, neuromuscular monitoring signal, cardiac output, venous flow, blood oxygen saturation (SpO₂), photo-plethysmograms (PPG) and/or electrocardiograms (ECG). In some embodiments, input signals can include transformed versions of signals (e.g., wavelet transforms).

In certain embodiments, inputs to signal generation processes can include additional data beyond the set of input signals. In several embodiments, inputs can include additional clinical information from the electronic health record (EHR) such as (but not limited to) demographics, lab results, vital signs, medications, health provider notes, omics data, touchless sensing, and/or features extracted from medical images. In various embodiments, other forms of input can be pre-processed (e.g., normalized to a common scale, aggregated, filtered, transformed, sequentialized, etc.). In a variety of embodiments, normalization can include subtracting the mean and dividing by the standard deviation. In many embodiments, clinical information for each patient can be aggregated into a standardized format and arranged into a sequence. Sequences of clinical data in accordance with many embodiments of the invention can then be combined with physiological waveform signals. In various embodiments, this sequence can be ordered chronologically and the combining operation can be done by matching the time of each waveform observation to co-occurring clinical events and observations.

Processes in accordance with some embodiments of the invention can augment inputs to a signal generation model. In a variety of embodiments, additional features can be generated for each point in time by calculating the mean and standard deviation of the non-invasive blood pressure measurements from the previous 15, 30, and 45 minutes. In certain embodiments, additional features can include non-invasive blood pressure (NIBP) that can be measured periodically (e.g., at 3 minute increments). As input waveforms are sampled at a much higher frequency (e.g., 100 Hz) than NIBP, processes in accordance with various embodiments of the invention can use forward filling interpolation to fill in missing NIBP values.

Processes in accordance with some embodiments of the invention can use a signal processing method (e.g., wavelet transform) to extract features of interest from various input signals (e.g., ECG, oscillometric waveforms, photo-plethysmographic (PPG), etc.). Rather than (or in addition to) using often-noisy raw ECG signals, processes in accordance with several embodiments of the invention can use a wavelet transform (e.g., with a Morlet wavelet (81.25 Hz)) to extract a smoothed version of the ECG signal. In certain embodiments, processes can use a different wavelet transform (e.g., a Mexican hat wavelet (e.g., 2.0833 Hz)) to extract the location of the T wave from the ECG signal. For photo-plethysmographic waveforms, processes in accordance with several embodiments of the invention can use wavelet transforms (e.g., a Mexican hat wavelet (e.g., 2.0833 Hz, 6.25 Hz) to derive the location of the dicrotic notch and/or to find the systolic peak. Frequencies for wavelet transforms in accordance with some embodiments of the invention can be chosen heuristically to detect certain signals (e.g., a QRS complex), while not detecting others (e.g., a P wave or T wave).

Process 100 processes (110) the set of input signals. Processing an input signal in accordance with some embodiments of the invention can include various pre-processing steps. In numerous embodiments, processing an input signal can include a variety of signal processing techniques, such as (but not limited to), filtering to reduce noise and remove outliers, and/or normalization to a common scale. Processes in accordance with numerous embodiments of the invention can process input signals differently based on an expected level of noise. For example, in certain embodiments processes can perform additional and/or stronger noise reduction processes in noisy environments (e.g., at a patient's home). In several embodiments, each input waveform signal can be downsampled (e.g., to 100 Hz (100 samples per second)) when the original sampling rate was greater than a sampling threshold (e.g., 100 Hz).

Since the range of different input signals (e.g., ECG and photo-plethysmographic signals) can differ for each patient, processes in accordance with various embodiments of the invention can scale each signal such that the magnitude is within the range of −1 to 1. However, because outliers caused by technical noise can result in skewed scaling, the scaling for outliers can be adjusted. An example of a process for scaling signal information is described in greater detail below.

Process 100 generates (115) an output signal. Output signals in accordance with several embodiments of the invention can include (but are not limited to) an arterial blood pressure (ABP) signal. In several embodiments, output signals can be generated with a signal generation model trained to produce the output signal from the inputs. Signal generation models in accordance with a variety of embodiments of the invention can take in various inputs including (but not limited to) time windows (e.g., 4 seconds) of a number of input signals (e.g., ECG, photo-plethysmogram, wavelet-transformed ECG, and/or wavelet-transformed photo-plethysmogram), the most recent non-invasive blood pressure measurement prior to the window, and/or statistics (e.g., mean, standard deviation) about the non-invasive blood pressure measurements taken during previous time intervals (e.g., 15, 30, and/or 45 minutes).

Generated outputs in accordance with some embodiments of the invention can include a time windowed (e.g., 4 second) arterial blood pressure waveform prediction, median systolic, median diastolic, and/or mean arterial blood pressure within the time window.

Process 100 provides (120) outputs based on the generated output signal. In numerous embodiments, processes can provide (either instead of, or in addition to the output signal) additional outputs such as (but not limited to) a visualization of the output signal, a summary statistic (e.g., blood pressure level), notifications, and/or alarms. Visualizations of the output signal can include (but are not limited to) displaying an imputed physiological waveform signal(s) in a graphical user interface (GUI) in real time. In several embodiments, imputed physiological waveforms can be used as inputs to other predictive algorithms. For example, imputed ABP waveforms in accordance with certain embodiments of the invention can be used to predict the probability (e.g., a risk score) that a patient will develop negative health outcomes (e.g., intraoperative hypotension). Predicted risk scores in accordance with certain embodiments of the invention can be displayed in a GUI.

While specific processes for generating imputed signals are described above, any of a variety of processes can be utilized to generate imputed signals as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted. Although the above embodiments of the invention are described in reference to imputing ABP signals, the techniques disclosed herein may be used in any type of signal generation, including [example alternative].

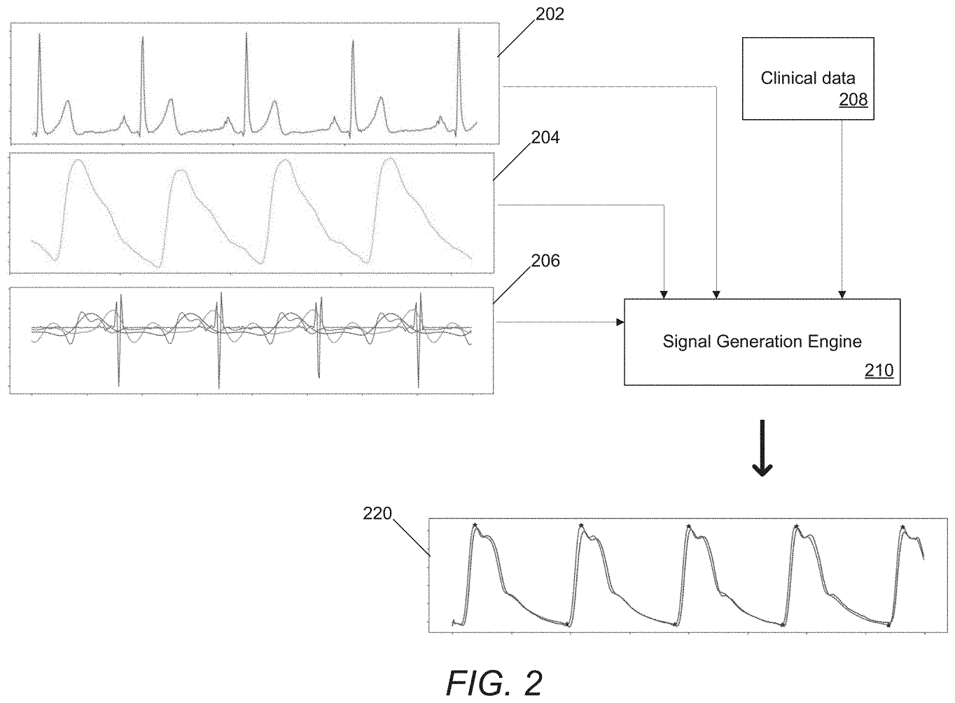

An illustration of the generation of ABP signals in accordance with an embodiment of the invention is illustrated in FIG. 2. In this example, various inputs 202-0208 are passed through a trained signal generation engine 210 to generate ABP signals 215. In this example, the inputs include ECG signals 202, PPG signals 204, transformed signals 206, and clinical data 208. Transformed signals in accordance with a number of embodiments of the invention can include signals that have been processed with wavelet transforms to emphasize particular characteristics of the signals. Clinical data in accordance with certain embodiments of the invention can include various patient information, such as (but not limited to) medical histories, personal characteristics, etc.

Inputs 202-208 are passed through signal generation engine 210 to generate a predicted continuous ABP signal 220.

While specific implementations of signal generation have been described above with respect to FIG. 2, there are numerous configurations, including, but not limited to, those using more, fewer, and/or different inputs or outputs, and/or any other configuration as appropriate to the requirements of a given application.

In several embodiments, processes can scale the entire record such that all values are in the range of −1 to 1, such that the smallest/largest outliers will become −1 and 1, but the rest of the signal can be compressed into a tiny range around 0. Processes in accordance with many embodiments of the invention can use medians, so that the resulting range will not be between −1 to 1 for the entire surgery, because outlier values will be above/below the −1 to 1 range. The goal of using the median min/max values of many windows in accordance with various embodiments of the invention is to get a distribution of min/max values. The median of such a distribution can be used as a way to remove the effect of large outliers that would otherwise skew the signal range.

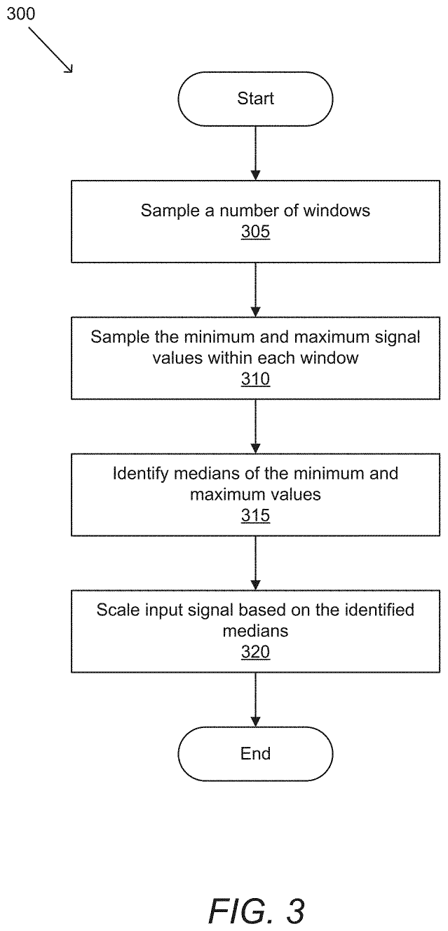

An example of a process for adjusting the scaling of waveform signals for outliers in accordance with an embodiment of the invention is conceptually illustrated in FIG. 3. In this example, process 300 samples (305) a number (e.g., 10,000) of windows of a set of input signals. Each sampled window in accordance with numerous embodiments of the invention is a particular duration (e.g., 4 seconds). In various embodiments, windows can be sampled in a variety of ways, including (but not limited to) randomly, in constant step times (e.g., every 2 seconds), etc.

Process 300 samples (310) the minimum and maximum signal values within each window and identifies (315) the medians of the minimum and maximum values. Process 300 scales (320) the signal based on the identified medians. In various embodiments, the signal can be scaled to a particular range (e.g., from −1 to 1).

While specific processes for scaling are described above, any of a variety of processes can be utilized to scale signals for outliers as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

In various embodiments, waveform signals can be processed as windows (e.g., 4 seconds) for training and/or generating new signals. For example, processes in accordance with numerous embodiments of the invention can pass windows of input waveform signals through a signal generation model to generate a window of output signal. Generated windows of output signal can be compared to a corresponding window of a true signal, and an error (or loss) for the generation can be passed back through the signal generation model to update the model. As signal processing can often be noisy and difficult, processes in accordance with a number of embodiments of the invention can process the signals to correct, filter, and/or scale the signals for use.

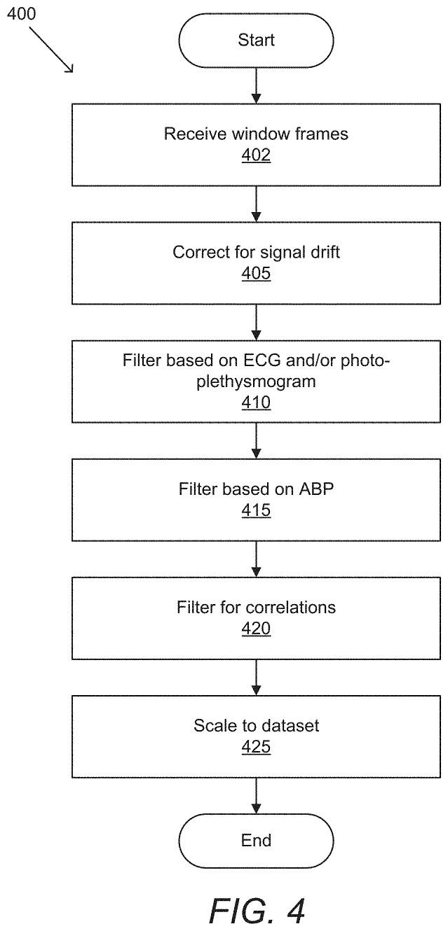

An example of a process for processing a set of signals in accordance with an embodiment of the invention is conceptually illustrated in FIG. 4. Process 400 receives (402) windows (or frames) of an input signal(s). In numerous embodiments, windows (e.g., 4 seconds) can be selected using a sliding window approach with a given (e.g., 2 second) step size. For each window, a filtering process in accordance with a number of embodiments of the invention can be used to determine whether the window will be included in the development or validation of the algorithm.

Process 400 corrects (405) for signal drift between different signals. In several embodiments, signal drift between a photo-plethysmographic signal and an arterial blood pressure (ABP) signal can be corrected. Processes in accordance with a variety of embodiments of the invention can correct signal drift by computing the cross-correlation of a smaller (e.g., 4 second) window of a signal to be corrected (e.g., photo-plethysmogram) under consideration with a larger (e.g., 32 second) overlapping window of another signal (e.g., ABP). In many embodiments, the larger window can be centered on the smaller window under consideration, and begins a period of time (e.g., 14 seconds) prior to the start of the smaller window. In various embodiments, once the cross-correlation is computed, the location of the highest cross-correlation can be used to correct the signal drift by shifting the signal to be corrected (e.g., photo-plethysmogram).

In several embodiments, waveform signals (e.g., ECG, PPG, arterial blood pressure, etc.) can be checked (or filtered) for signal quality to remove windows with technical artifacts or invalid parameters. In certain embodiments, windows of a signal can be excluded based on various filtering criteria. Process 400 filters (410) based on ECG and/or photo-plethysmogram (PPG). Filtering criteria in accordance with certain embodiments of the invention for the ECG or photo-plethysmogram data can include (but are not limited to) one or more of whether: the minimum or maximum signal value in a window exceeds a minimum or maximum threshold (e.g., 7.0 for ECG and plethysmogram), variance of a signal is less than a variance threshold (e.g., 1e-4 for ECG, 1e-2 for photo-plethysmogram), the number of peaks in window exceeds a maximum peak threshold (e.g., 4 peaks/sec×4 seconds for both ECG, photo-plethysmogram), and/or a number of peaks in window is less than a minimum peak threshold (e.g., 0.5 peak/sec×4 seconds for both ECG, photo-plethysmogram).

Process 400 filters (415) based on ABP. For the arterial blood pressure waveform, filtering criteria in accordance with numerous embodiments of the invention can include (but are not limited to) one or more of whether: mean signal value is outside of a mean signal range (e.g., less than 30 mmHg or greater than 200 mmHg), maximum signal value is outside of a maximum signal range (e.g., greater than 300 mmHg or less than 60 mmHg), minimum signal value is less than a minimum signal threshold (e.g., 20 mmHg), variance of a signal is less than a variance threshold (e.g., 80), systolic or diastolic blood pressure values cannot be found using a peak finding algorithm (e.g., the find_peaks function from the SciPy package), a difference between two consecutive systolic or diastolic values is greater than 50 mmHg, and/or a time delay between diastolic blood pressure measurement and systolic blood pressure measurement was greater than a delay threshold (e.g., 0.5 seconds).

Process 400 filters (420) for correlations between the different signals. Correlation filtering criteria in accordance with several embodiments of the invention can include (but are not limited to) one or more of whether: a number of photo-plethysmogram peaks is different than the number of arterial blood pressure peaks, and/or a mean absolute time difference (after signal drift correction) between arterial blood pressure peaks and photo-plethysmogram peaks was greater than a threshold duration (e.g., 0.15 seconds).

Once the frames have been filtered according to various filtering criteria, process 400 scales (425) the remaining frames. In a variety of embodiments, using a running mean and standard deviation, the ECG, photo-plethysmogram, and the wavelet transforms of each of the signals can be scaled (e.g., to have a mean of zero and standard deviation of one). In many embodiments, different methods of scaling the remaining frames can be used without departing from the invention.

While specific processes for filtering and processing signals are described above, any of a variety of processes can be utilized to filter and process as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted. Although many of the examples described herein processing of ECG, photo-plethysmogram, and/or ABP signals, one skilled in the art will recognize that similar systems and methods can be used in a variety of applications, including (but not limited to) training a signal generation model and/or generating an imputed signal, without departing from this invention.

### Network Architecture

In a number of embodiments, processes in accordance with many embodiments of the invention can implement deep learning models. Models in accordance with various embodiments of the invention can include (but are not limited to) convolutional neural networks (CNNs), long-short term memory (LSTM) networks, and/or recurrent neural networks (RNNs).

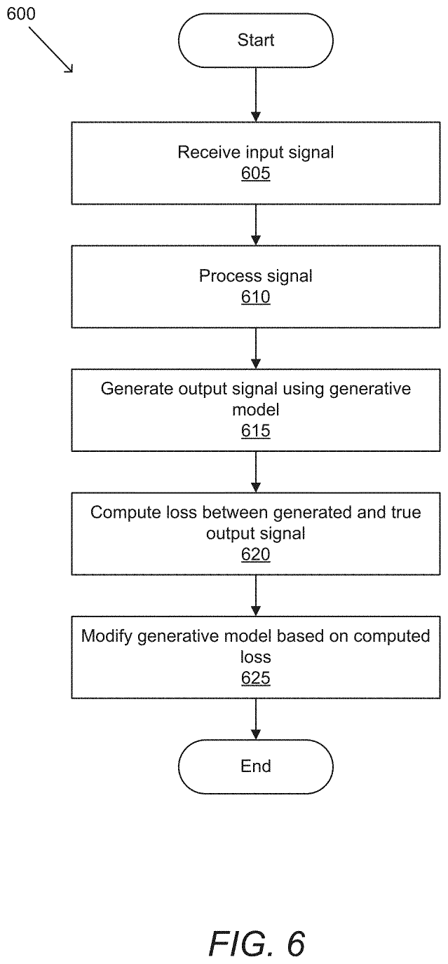

To learn the sequence-to-sequence mapping, models in accordance with a variety of embodiments of the invention can be trained on physiological waveform data and clinical data to impute a set of physiological waveform signals by reducing a loss function. Loss functions can measure the difference between the imputed signal and the true signal. Additionally, loss functions can incorporate measures that are important for the clinical interpretation of the waveform. Training with loss functions in accordance with certain embodiments of the invention is described in greater detail below with reference to FIG. 6.

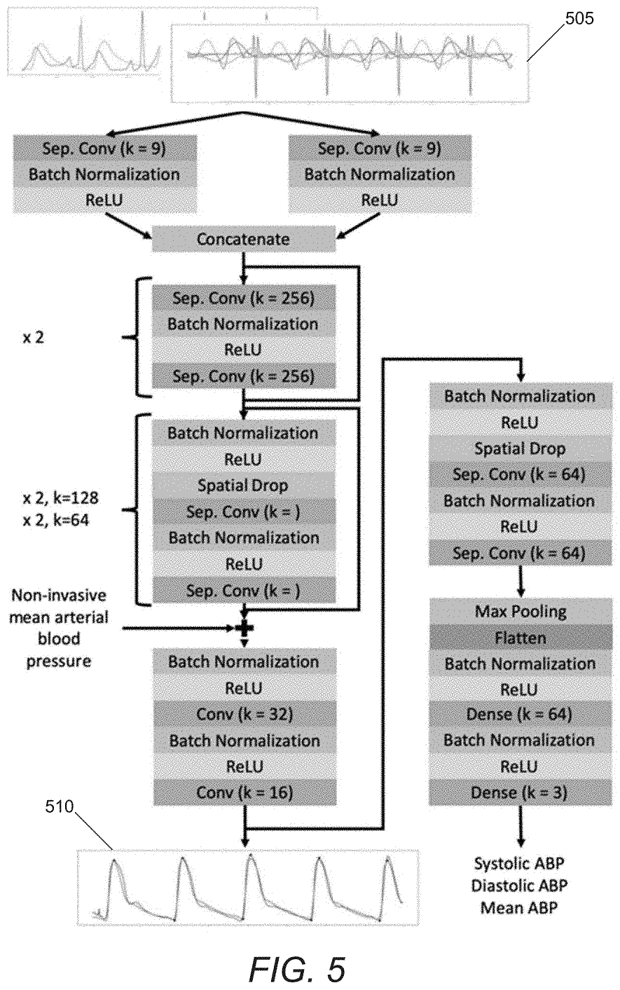

An example of a network architecture for a signal generation model in accordance with an embodiment of the invention is illustrated in FIG. 5. In this example, deep learning model 500 takes as input a 4 second window 505 of photo-plethysmogram, wavelet-transformed ECG, wavelet-transformed photo-plethysmogram, the most recent non-invasive blood pressure measurement prior to the window, and statistics (mean, standard deviation) about the non-invasive blood pressure measurements taken during previous time intervals (e.g., 15, 30, and/or 45 minutes). Deep learning models in accordance with some embodiments of the invention can be trained to output a time windowed (e.g., 4 second) arterial blood pressure waveform prediction 510, median systolic, median diastolic, and/or mean arterial blood pressure within the time window.

The network architecture of this example consists of a total of 18 convolutional layers, organized into blocks with skip connections between the start and the end of each block to improve the optimization procedure. Each block contains 2 separable convolutional layers, with filter widths of either 256, 128, or 64. Prior to convolutional layers, batch normalization and a rectified linear unit were applied. Spatial dropout layers were inserted between convolutional layers, with a dropout probability of 0.2.

The deep-learning model was trained using random weight initialization and the Nadam optimizer with parameters beta1 of 0.9, beta2 of 0.999, a schedule decay of 0.004, and clipnorm of 0.5. The learning rate used was 0.002, and the mini batch size was 64. Network architecture hyperparameters were chosen using manual tuning.

While specific implementations of signal generation architectures have been described above with respect to 5, there are numerous configurations of signal generation architectures, including, but not limited to, those using other CNNs, RNNs, LSTM networks, and/or any other configuration as appropriate to the requirements of a given application.

In many embodiments, signal generation architectures can include encoder-decoder architectures (e.g., the V-Net architecture, has been proven to be an effective method for segmentation of 2D and 3D images) that can be used for 1D signal-to-signal transformation. Signal generation architectures in accordance with many embodiments of the invention can learn a compressed representation of the input data to identify global features, and then reconstruct the signal from this representation. Signal generation architectures in accordance with numerous embodiments of the invention can include a compression stage, where the image resolution is consecutively reduced using a number of convolutional layers (downsampling), and a decompression stage, where the image resolution is recovered using the same number of de-convolutional layers (upsampling). Compression stages in accordance with a number of embodiments of the invention can allow the network to learn global features in the compressed representation. Decompression stages in accordance with certain embodiments of the invention can allow the network to learn to localize the features that were identified in the compressed representation. In a number of embodiments, compression stages can reduce the resolution by using a kernel stride size of two, which effectively downsamples the signal by a factor of two (similar to a traditional pooling operation used in CNNs). While the signal is downsampled by a factor of two, the number of features (channels) extracted increases by a factor of two.

In several embodiments, residual connections can be used between the convolutional and de-convolutional layers at the same depth of each stage. Residual connections in accordance with certain embodiments of the invention can force the network to learn a residual function, which can accelerate the convergence process. Use of residual connections in accordance with a number of embodiments of the invention is further motivated by the similarity of the PPG waveform to the ABP waveform, as demonstrated by the performance of the PPG scaling method. Since the shape of the two waveforms is relatively similar, models in accordance with various embodiments of the invention can learn to predict the difference rather than learn a more complex transformation of the PPG waveform that matches the ABP waveform. In certain embodiments, rather than solely rely on the PPG waveform for predicting the ABP waveform, models can incorporate features extracted from the EKG waveform. This serves two primary purposes: it can provide additional information to supplement the PPG waveform, and can allow the method to be more robust to signal artifacts that may occur in one or both waveforms, by leveraging a combination of the two.

In various embodiments, signal generation architectures can be similar to that as described in described in “V-Net: Fully Convolutional Neural Networks for Volumetric Medical Image Segmentation” by Milletari et al., the disclosure of which is incorporated by reference herein in its entirety. However, rather than 3D volumes with multiple channels, processes in accordance with several embodiments of the invention can represent data as a 1D signal with multiple channels. One skilled in the art will recognize that similar architectures can be used without departing from this invention. For example, in certain embodiments, signal generation architectures can use twice the number of channels at each layer in the network.

### Training Signal Generation Models

Signal generation models in accordance with several embodiments of the invention can be trained to generate a physiological waveform signal based on other physiological waveform signals. An example of a process for training a signal generation model in accordance with an embodiment of the invention is conceptually illustrated in FIG. 6. Process 600 receives (605) an input signal. Input signals in accordance with many embodiments of the invention can include non-invasive signals that are monitored during a surgical procedure. In various embodiments, input signals can include (but are not limited to) blood pressure, heart rates, blood oxygen saturation (SpO₂), photo-plethysmograms (PPG) and/or electrocardiograms (ECG). In a variety of embodiments, inputs can also include other additional data, such as (but not limited to) clinical information, summary statistics, transformed signals, etc. In addition to the input signals, processes in accordance with various embodiments of the invention can receive output training data. Output training data in accordance with some embodiments of the invention can include desired output data, such as (but not limited to) invasive signals (e.g., ABP).

Process 600 processes (610) the received signal. Processing the signal in accordance with numerous embodiments of the invention can include various signal processing methods, such as (but not limited to) those described throughout this description.

Process 600 generates (615) an output signal using a generative model. In numerous embodiments, non-invasive ECG and photo-plethysmogram waveforms and clinical data can be used as input to predict the arterial blood pressure (ABP) waveform, which would often be obtained invasively. In various embodiments, generative models are initialized with random weights, which can be modified and updated based on computed losses through the training process.

Process 600 computes (620) a loss between the generated output signal and a true output signal. Loss functions in accordance with various embodiments of the invention can be computed as an average difference between the imputed ABP waveform and the true ABP waveform. In a number of embodiments, loss functions can include an additional penalty for the difference between the maximum and minimum signal points (corresponding to the systolic and diastolic blood pressure) in the imputed and true waveforms. True output signals in accordance with a number of embodiments of the invention can be recorded using an invasive technique, allowing the model to learn to impute the invasive signal.

Process 600 modifies (625) the generative model based on the computed loss. In a variety of embodiments, weights of a generative model can be modified through a backpropagation process that passes a computed loss back through layers of a generative model.

In several embodiments, processes can further calibrate a trained model to generate more accurate signals for a subject. In many embodiments, processes can calibrate results as the signal is generated, readjusting the results based on periodic cuff readings (e.g., every few minutes). Calibration in accordance with numerous embodiments of the invention can be performed using a series of more frequent cuff measurements (e.g., over 20-30 minutes) for a particular individual, which can be used to determine the mean and variance of the blood pressure over the extended time period and the corresponding values in the PPG and ECG signals. In a variety of embodiments, a trained model can be calibrated based on differences between predictions of the trained model and the series of actual cuff measurements for an individual.

While specific processes for training a signal generation model are described above, any of a variety of processes can be utilized to train such models as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Results

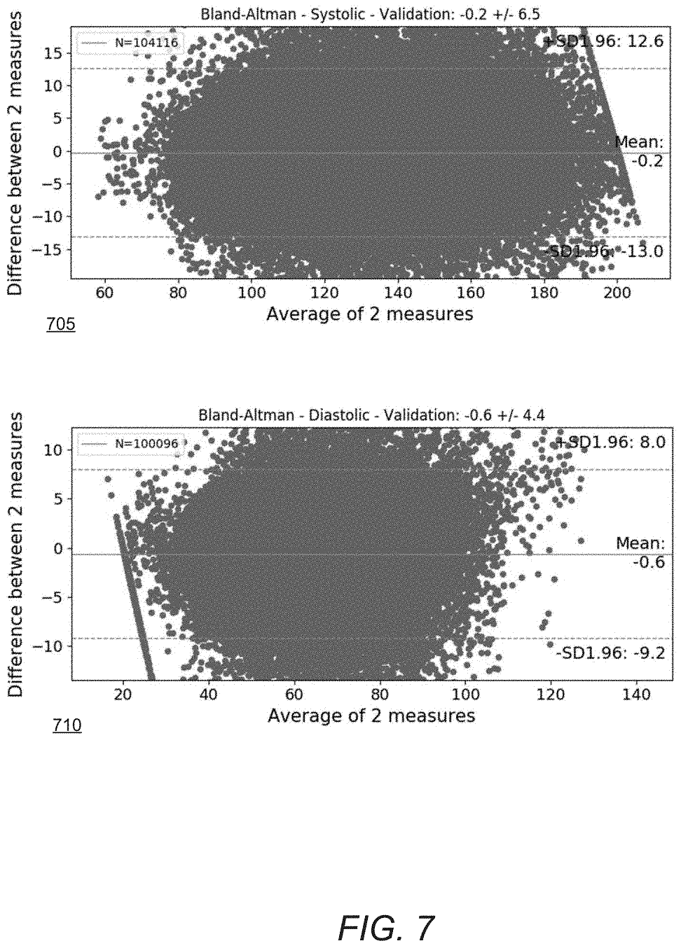

To evaluate the agreement between the gold standard blood pressure measurements (the arterial catheter) and the deep neural network (DNN) predictions, the Bland and Altman method was used. The method was implemented as follows. For each 4 second window under consideration, systolic and diastolic blood pressure measurements were extracted from the arterial blood pressure waveform using a peak finding algorithm. These measurements were used as the reference values. At the same time points in the 4 second window, DNN-predicted blood pressure values were extracted from the generated waveform as comparison values. The difference between the reference blood pressure measurements and the predicted blood pressure measurements were plotted as a function of the average of the reference and predicted value pairs. A visualization of plots for the differences between the predicted and actual measures is illustrated in FIG. 7. In this figure the first chart 705 illustrates the differences for the predicted systolic values, while the second chart 710 illustrates the differences for the predicted diastolic values.

### Systems for Doing Something

### Signal Generation System

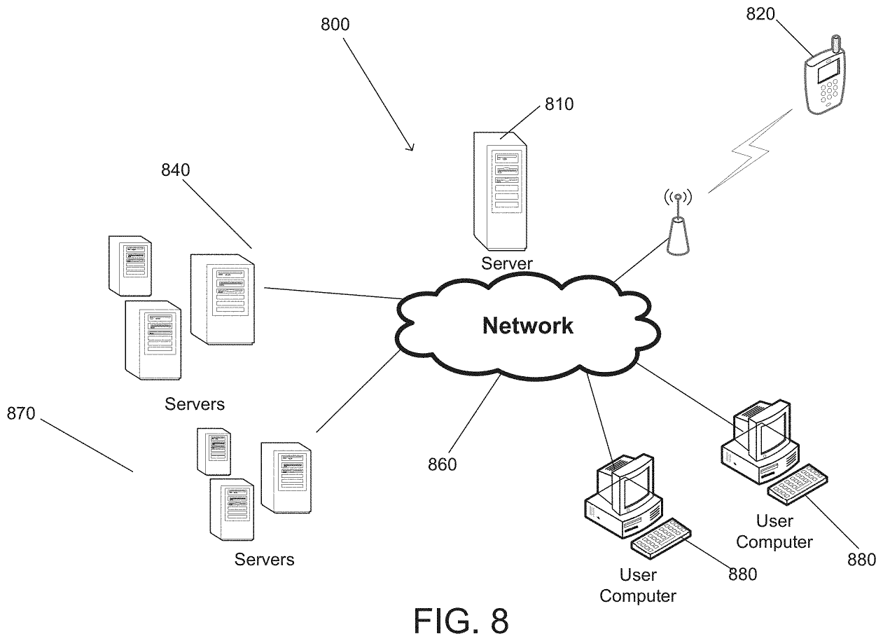

A signal generation system that imputes waveform signals in accordance with some embodiments of the invention is shown in FIG. 8. Network 800 includes a communications network 860. The communications network 860 is a network such as the Internet that allows devices connected to the network 860 to communicate with other connected devices. Server systems 810, 840, and 870 are connected to the network 860. Each of the server systems 810, 840, and 870 is a group of one or more servers communicatively connected to one another via internal networks that execute processes that provide cloud services to users over the network 860. For purposes of this discussion, cloud services are one or more applications that are executed by one or more server systems to provide data and/or executable applications to devices over a network. The server systems 810, 840, and 870 are shown each having three servers in the internal network. However, the server systems 810, 840 and 870 may include any number of servers and any additional number of server systems may be connected to the network 860 to provide cloud services. In accordance with various embodiments of this invention, a signal generation system that uses systems and methods that train signal generation models and/or generate waveform signals in accordance with an embodiment of the invention may be provided by a process being executed on a single server system and/or a group of server systems communicating over network 860.

Users may use personal devices 880 and 820 that connect to the network 860 to perform processes that train signal generation models and/or generate waveform signals in accordance with various embodiments of the invention. Personal devices in accordance with several embodiments of the invention can include various devices with sensors to measure and record signals, which can be used to generate physiological waveform signals (e.g., on the device, in the cloud, etc.). In the shown embodiment, the personal devices 880 are shown as desktop computers that are connected via a conventional “wired” connection to the network 860. However, the personal device 880 may be a desktop computer, a laptop computer, a smart television, an entertainment gaming console, or any other device that connects to the network 860 via a “wired” connection. The mobile device 820 connects to network 860 using a wireless connection. A wireless connection is a connection that uses Radio Frequency (RF) signals, Infrared signals, or any other form of wireless signaling to connect to the network 860. In FIG. 8, the mobile device 820 is a mobile telephone. However, mobile device 820 may be a mobile phone, Personal Digital Assistant (PDA), a tablet, a smartphone, or any other type of device that connects to network 860 via wireless connection without departing from this invention.

As can readily be appreciated the specific computing system used to train signal generation models and/or generate waveform signals is largely dependent upon the requirements of a given application and should not be considered as limited to any specific computing system(s) implementation.

### Signal Generation Element

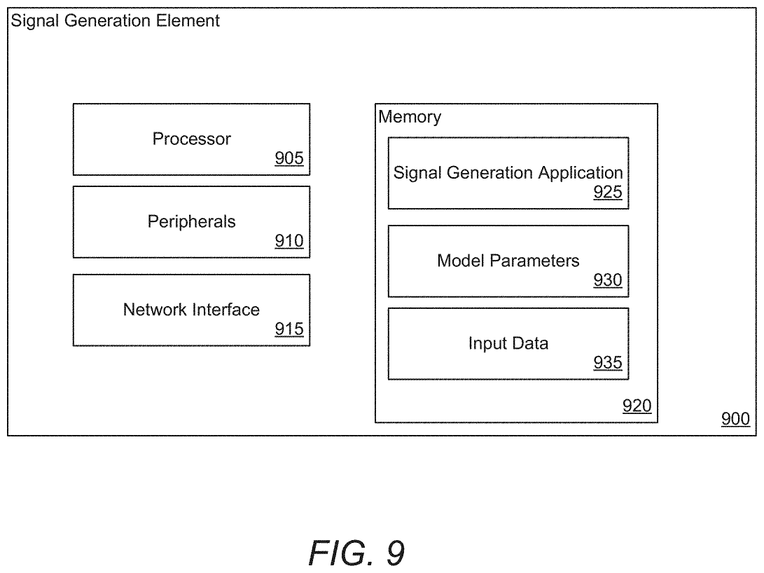

An example of a signal generation element that executes instructions to perform processes that can generate waveform signals in accordance with various embodiments of the invention is shown in FIG. 9. Signal generation elements in accordance with many embodiments of the invention can include (but are not limited to) one or more of mobile devices, medical devices, cameras, and/or computers. In numerous embodiments, signal generation elements can be operated in various settings, such as at a hospital, in a doctor's office, in a home setting, etc. Signal generation elements in accordance with certain embodiments of the invention can use varying numbers of ECG leads for different applications. For example, in some embodiments, signal generation elements can use fewer ECG leads (e.g., 10) for home applications, while using a higher number of leads (e.g., 12) for hospital use. Signal generation element 900 includes processor 905, peripherals 910, network interface 915, and memory 920.

One skilled in the art will recognize that a particular signal generation element may include other components that are omitted for brevity without departing from this invention. The processor 905 can include (but is not limited to) a processor, microprocessor, controller, or a combination of processors, microprocessor, and/or controllers that performs instructions stored in the memory 920 to manipulate data stored in the memory. Processor instructions can configure the processor 905 to perform signal generation processes in accordance with certain embodiments of the invention.

Peripherals 910 can include any of a variety of components for capturing data, such as (but not limited to) cameras, displays, and/or sensors. In a variety of embodiments, peripherals can be used to gather inputs and/or provide outputs. Network interface 915 allows signal generation element 900 to transmit and receive data over a network based upon the instructions performed by processor 905. Peripherals and/or network interfaces in accordance with many embodiments of the invention can be used to gather inputs that can be used to impute physiological waveform signals.

Memory 920 includes a signal generation application 925, model parameters 930, and input data 935. Signal generation applications in accordance with several embodiments of the invention can be used to generate real-time physiological waveform signals based on a set of input signals. In many embodiments, signal generation applications can be used to impute ABP signals from ECG and/or PPG signals.

Although a specific example of a signal generation element 900 is illustrated in FIG. 9, any of a variety of signal generation elements can be utilized to perform processes for generating waveform signals similar to those described herein as appropriate to the requirements of specific applications in accordance with embodiments of the invention.

### Training Element

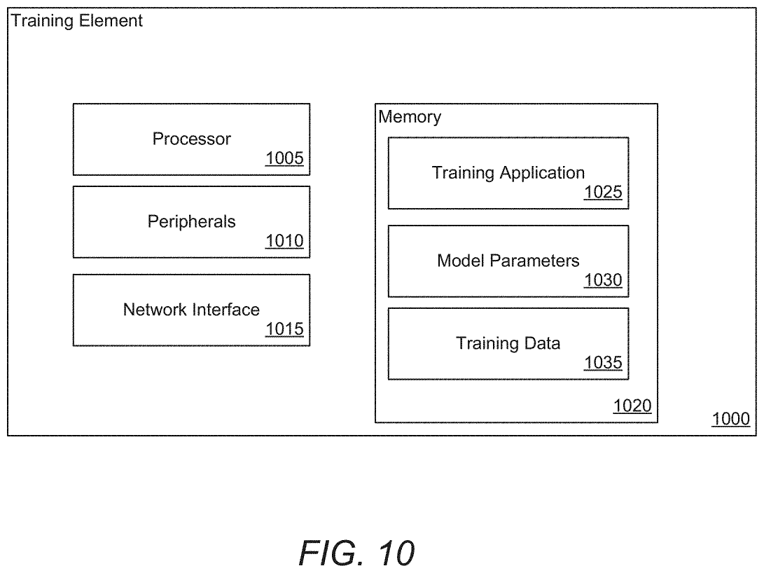

An example of a training element that executes instructions to perform processes that can train signal generation systems in accordance with various embodiments of the invention is shown in FIG. 10. Training elements in accordance with many embodiments of the invention can include (but are not limited to) one or more of mobile devices, servers, cloud services, and/or computers. Training element 1000 includes processor 1005, peripherals 1010, network interface 1015, and memory 1020.

One skilled in the art will recognize that a particular training element may include other components that are omitted for brevity without departing from this invention. The processor 1005 can include (but is not limited to) a processor, microprocessor, controller, or a combination of processors, microprocessor, and/or controllers that performs instructions stored in the memory 1020 to manipulate data stored in the memory. Processor instructions can configure the processor 1005 to perform training processes in accordance with certain embodiments of the invention.

Peripherals 1010 can include any of a variety of components for capturing data, such as (but not limited to) cameras, displays, and/or sensors. In a variety of embodiments, peripherals can be used to gather inputs and/or provide outputs. Network interface 1015 allows training element 1000 to transmit and receive data over a network based upon the instructions performed by processor 1005. Peripherals and/or network interfaces in accordance with many embodiments of the invention can be used to gather inputs that can be used to train a signal generation model. Inputs in accordance with a number of embodiments of the invention can include physiological waveform signals from various medical devices.

Memory 1020 includes a training application 1025, model parameters 1030, and training data 1035. Training applications in accordance with several embodiments of the invention can be used to train a signal generation model.

Although a specific example of a training element 1000 is illustrated in FIG. 10, any of a variety of training elements can be utilized to perform processes for training signal generation models similar to those described herein as appropriate to the requirements of specific applications in accordance with embodiments of the invention.

### Signal Generation Application

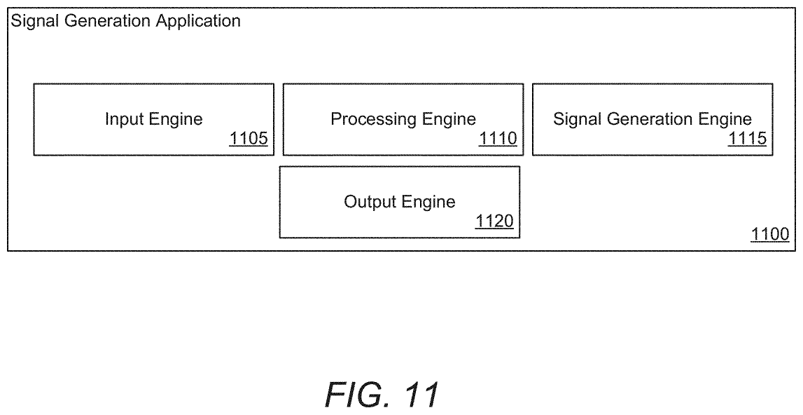

A signal generation application for generating waveform signals in accordance with an embodiment of the invention is illustrated in FIG. 11. Signal generation application 1100 includes input engine 1105, processing engine 1110, signal generation engine 1115, and output engine 1120.

Input engines in accordance with some embodiments of the invention can receive inputs from various different sources, such as (but not limited to) medical devices (e.g., electrocardiograph, photo-plethysmographs, heart rate monitors, etc.), network servers, electronic health records, and/or manual inputs.

In several embodiments, processing engines can pre-process inputs in a variety of ways, including (but not limited to) signal processing, filtering, scaling, normalization, signal drift correction, wavelet transforms, downsampling, etc. Processing engines can be used to clean and prepare inputs to a signal generation engine.

Signal generation engines in accordance with a number of embodiments of the invention can take inputs and generate a physiological waveform signal (e.g., ABP) based on the inputs. In several embodiments, signal generation engines include a deep learning sequence-to-sequence model that is trained to generate a corresponding output signal from an input sequence of inputs.

In various embodiments, output engines can provide a variety of outputs to a user, including (but not limited to) real-time, continuous physiological waveform signals. Output engines in accordance with various embodiments of the invention can provide summary statistics (e.g., blood pressure levels), notifications, and alarms. In a variety of embodiments, output engines can provide an output (e.g., an alert) when an output waveform signal (and/or a summary statistic) exceeds a threshold. In many embodiments, output engines can provide data for a generated waveform signal to a predictive engine, which can be used to predict a probability for various health outcomes.

### Training Application

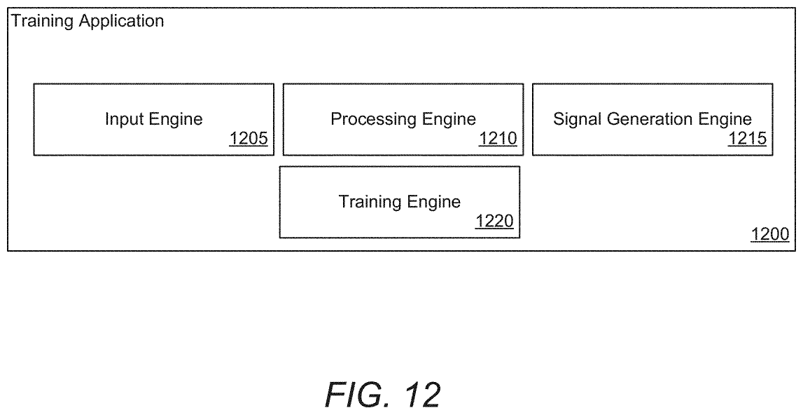

A training application for training signal generation models in accordance with an embodiment of the invention is illustrated in FIG. 12. Training application 1200 includes input engine 1205, processing engine 1210, signal generation engine 1215, and training engine 1220. In many embodiments, input engines, processing engines, and signal generation engines are similar to those of a signal generation application, such as the example described above with reference to FIG. 11.

Signal generation engines in accordance with several embodiments of the invention can be initialized with random weights and/or parameters, which can be adjusted as the model is trained. In several embodiments, training engines can compute a set of one or more losses. Loss functions in accordance with various embodiments of the invention can be computed as an average difference between the imputed ABP waveform and the true ABP waveform. In a number of embodiments, loss functions can include an additional penalty for the difference between the maximum and minimum signal points (corresponding to the systolic and diastolic blood pressure) in the imputed and true waveforms.

Training engines in accordance with numerous embodiments of the invention can pass computed losses back through signal generation models to update the weights and train the model to generate more accurate signals. Trained signal generation engines in accordance with some embodiments of the invention can be used to simulate invasive physiological waveform signals based on non-invasive input signals.

Although specific methods of training signal generation models and/or generating waveform signals are discussed above, many different methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for measuring physiological waveform signals, the method comprising:
receiving a set of one or more input physiological waveform signals;
processing the set of input physiological waveform signals;
generating an output physiological waveform signal using a signal generation model; and
providing outputs based on the generated output signal.

2. The method of claim 1, where receiving the set of input physiological waveform signals comprises capturing the set of input signals in a non-invasive manner.

3. The method of claim 1, where processing the set of input physiological waveform signals comprises at least one of normalizing, scaling, filtering, and downsampling the set of input physiological waveform signals.

4. The method of claim 1, where the signal generation model is a convolutional neural network (CNN) that takes a set of windows from the set of input physiological waveform signals as input and generates a window of the output physiological waveform signal.

5. The method of claim 1, where providing outputs comprises providing at least one of a summary statistic and a visualization of the output physiological waveform signal.

6. A method for training a signal generation model to generate a physiological waveform signal, the method comprising:
receiving a set of one or more input physiological waveform signals;
processing the set of input physiological waveform signals;
generating an output physiological waveform signal using a signal generation model;
computing a loss between the generated output physiological waveform signal and a true output physiological waveform signal; and
modifying the signal generation model based on the computed loss.

7. The method of claim 6, where the set of input physiological waveform signals comprises at least one of an electrocardiogram (ECG) and a photo-plethysmogram (PPG).

8. The method of claim 6, where processing the set of input physiological waveform signals comprises at least one of normalizing, scaling, filtering, and downsampling the set of input physiological waveform signals.

9. The method of claim 6, where the signal generation model is a convolutional neural network (CNN) initialized with a random set of weights.

10. The method of claim 6, where computing a loss comprises a penalty for the difference between maximum signal points and minimum signal points of the output and true physiological waveform signals.

11. A non-transitory machine readable medium containing processor instructions for measuring physiological waveform signals, where execution of the instructions by a processor causes the processor to perform a process that comprises:
receiving a set of one or more input physiological waveform signals;
processing the set of input physiological waveform signals;
generating an output physiological waveform signal using a signal generation model; and
providing outputs based on the generated output signal.

12. The non-transitory machine readable medium of claim 11, where receiving the set of input physiological waveform signals comprises capturing the set of input signals in a non-invasive manner.

13. The non-transitory machine readable medium of claim 11, where processing the set of input physiological waveform signals comprises at least one of normalizing, scaling, filtering, and downsampling the set of input physiological waveform signals.

14. The non-transitory machine readable medium of claim 11, where the signal generation model is a convolutional neural network (CNN) that takes a set of windows from the set of input physiological waveform signals as input and generates a window of the output physiological waveform signal.

15. The non-transitory machine readable medium of claim 11, where providing outputs comprises providing at least one of a summary statistic and a visualization of the output physiological waveform signal.

16. The non-transitory machine readable medium of claim 11, wherein:
the process further comprises training the signal generation model using data from a plurality of individuals; and
the input physiological waveform signals are from an individual that is not included in the plurality of individuals.

17. The non-transitory machine readable medium of claim 11, wherein processing the set of input physiological waveform signals comprises performing a noise-reduction process on the input physiological waveform signals.
